(19) United States
(12) Patent Application Publication
(10) Pub. No.: US 2020/0386734 A1
(43) Pub. Date: Dec. 10, 2020

(54) PLANT SUBSTRATE SENSOR STATION

(71) Applicant: METER Group, Inc. USA, Pullman, WA (US)

(72) Inventors: Manuel Irritier, Munich (DE); Evan Elbek, Pullman, WA (US)

(73) Assignee: METER Group, Inc. USA, Pullman, WA (US)

(21) Appl. No.: 16/431,998

(22) Filed: Jun. 5, 2019

(51) Int. Cl. G01N 33/24

(52) U.S. Cl. CPC G01N 33/24; G01N 33/246; G01N 2033/245

(57) ABSTRACT

Sensor stations for horticulture and other plant cultivation which include a base housing and a vertically-oriented sensor configured to measure properties of a plant substrate material. Some sensor stations have a set of finger portions or support arms to orient and retain the substrate material relative to the sensor station. Sensors are more reliably and consistently positioned within plant substrates when using the sensor stations. The sensor stations also have features improving resistance to damage or interference from fluids and debris. The sensor station is electrically connectable to a network and other sensor stations to more efficiently and securely transfer measurement data and instructions.

### TECHNICAL FIELD

The present disclosure generally relates to agricultural and horticultural products and methods for testing and monitoring plant growth conditions.

### BACKGROUND

Modern horticulture and related techniques implement sensors for collecting and monitoring measurements of water content, electrical conductivity, temperature, pH, drainage volume, and other properties in blocks of substrate material. In many cases, sensors and probes are embedded in the substrate material to facilitate continuous monitoring and updates of the sensor data. Data from the sensors is then used to control irrigation schedules, fertilizer schedules, the pH and temperature of water provided to the plant, and other related steps. Proper measurement of the substrate properties improves efficiency by saving resources and improving growth outcomes.

Generally, the sensors used with horticulture substrates are not optimized to use with these substrates and are instead intended for installation in bulk soil or liquid water. Horticulture substrate materials such as coco coir, peat, perlite, stonewool, and mixtures thereof present challenges and opportunities for growers that are not well addressed by sensors made for installation in bulk soil and liquid water in part because of their shape, size, and material properties. Accordingly, there is a constant need for improvements to horticulture equipment and sensors.

### SUMMARY

Aspects of the present disclosure relate to a plant substrate sensor station. The station can comprise a housing including a platform to contact a vertically-facing surface of a plant substrate material mounted to the housing and a sensor retainer to retain a sensor probe in a vertical orientation through the vertically-facing surface of the plant substrate material mounted to the housing. The station can also include an electronics station mounted to the housing and configured to receive a signal from the sensor probe.

In some embodiments, the station further includes the sensor probe retained by the sensor retainer, with the sensor probe electrically connected to the electronics station, the sensor probe extending vertically into a space adjacent to the platform, and the space being configured to be occupied by the plant substrate material. A wireless transceiver can be connected to the electronics station to transmit the signal from the sensor probe. A renewable power generator can be provided to power to the sensor probe, the electronics station, and the wireless transceiver, with the renewable power generator being mounted to the housing, laterally spaced from the platform, and configured to be laterally spaced away from the vertically-facing surface of the plant substrate material when the plant substrate material is mounted to the housing.

The station can also further comprise the sensor probe, with the sensor probe being retained by the sensor retainer, and with the sensor probe having an elongated sensor prong extending vertically from the sensor retainer to penetrate the plant substrate material. The housing can comprise a base portion having a second vertically-facing surface with the platform being vertically spaced away from the second vertically-facing surface. The platform can comprise a set of spaced apart posts to contact the plant substrate material, and the sensor retainer can be configured to retain the sensor probe centered within the set of spaced apart posts.

In some embodiments, the housing includes a finger portion to engage a lateral side surface of the plant substrate material. The housing can also include a second finger portion to engage a second lateral side surface of the plant substrate material. The housing can include a top surface having a channel positioned between the platform and the electronics station. The platform can be attachable to the housing in at least two discrete positions relative to the sensor retainer.

In another aspect of the disclosure, a plant substrate sensor station is provided, wherein the station includes a base platform. A plant substrate material is contactable by the base platform while the plant substrate material is in a substrate support zone adjoining the base platform. The station can also have a sensor probe mounted to the base platform, with the sensor probe having an elongated transducer extending perpendicular to the base platform and vertically into the substrate support zone.

The station can further comprise an electronic receiver in electrical communication with the sensor probe or a set of finger portions configured to extend alongside the plant substrate material in a direction substantially perpendicular to the base platform. The base platform can be configured to be centered under the plant substrate material and the elongated transducer can be positioned within the base platform. The sensor probe can be centered within the base platform.

In yet another aspect of the disclosure, a plant substrate sensor station is provided, wherein the station comprises a base housing having a top surface configured to be positioned beneath a plant substrate material, with the plant substrate material having an outer perimeter and a bottom surface, a substrate support stand having a first support surface and a second support surface, with the first support surface being configured to contact a first side surface of the plant substrate material, with the second support surface being configured to contact a second side surface of the plant substrate material, and with the first side surface being opposite the second side surface, and a sensor system configured to measure properties of the plant substrate material while the plant substrate material is contacted by the first support surface and the second support surface.

In some embodiments, the substrate support stand comprises a set of posts, the set of posts comprising a first post having the first support surface and a second post having the second support surface. A block of the plant substrate material can be vertically insertable into the substrate support stand. The substrate support stand can comprise a set of platform portions configured to support corners of the plant substrate material. The set of platform portions can be vertically spaced away from the top surface of the base housing.

The above summary of the present invention is not intended to describe each embodiment or every implementation of the present invention. The Figures and the detailed description that follow more particularly exemplify one or more preferred embodiments.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIGS. 1A-1F are side views of various embodiments of substrate sensor stations according to the present disclosure.

FIG. 2A is an isometric view of a substrate sensor station of the present disclosure.

FIG. 2B is an isometric view of a substrate sensor station of the present disclosure.

FIG. 2C is a top view of a substrate sensor station of the present disclosure.

FIG. 2D is a front view of a substrate sensor station of the present disclosure.

FIG. 2E is a side view of a substrate sensor station of the present disclosure.

FIG. 3 is an exploded view of a substrate sensor station of the present disclosure.

FIG. 4 is a second configuration of the substrate sensor station of FIG. 2A.

FIG. 5 is an isometric view of a substrate sensor station of the present disclosure.

FIG. 6 is an isometric view of a substrate sensor station of the present disclosure.

FIG. 7 is a schematic network diagram of a system of the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure generally relates to sensor equipment and related systems and methods for monitoring and measuring properties of plants and plant substrates such as horticulture substrates. Materials used for substrates generally exhibit strong vertical gradients and horizontal gradients in their internal properties. Thus, the water content, electrical conductivity, temperature, pH, drainage volume, or other properties of the substrate can greatly vary based on the vertical depth or horizontal position at which the measurement is taken. Accordingly, cultivators can obtain unreliable or inconsistent data (or aggregations of data) because of inconsistencies in sensor placement from plant to plant, substrate to substrate, and pot to pot. Embodiments of the present disclosure can improve the consistency and precision of sensor installation by managing the insertion position and depth of a sensor probe, especially in substrates such as stonewool cubes which have extreme vertical gradients in water content or bagged coir and similar loose and unconsolidated materials. Using embodiments of the present disclosure, horticulture laborers are less reliant on time-consuming (and therefore expensive) processes that require measuring devices (e.g., rulers and tape measures) and human judgement to keep sensor placement reliably consistent from substrate to substrate. By comparison, conventional practices can be burdensome to laborers when sensors are removed and then reinstalled in what can be dirty, entangling, and disorganized workspaces.

Additionally, although many commercially-available sensors connect to a data recording and storage device with a cable to provide communication and power to the sensor, the cable can obstruct laborers' work, can be damaged when moved, and can require unnecessarily burdensome effort to install and maintain. Those systems can also require multi-port data loggers that limit the user's flexibility when deciding where to locate sensors within a horticulture facility in order to optimize sample size and location. Aspects of the present disclosure can improve upon those devices by implementing wireless communication transceivers and alternative power sources that simultaneously save energy, decrease the difficulty of installation of multiple stations, and increase the portability of the station.

Aspects of the present disclosure relate to a self-contained sensing platform with embedded sensors that is capable of holding substrates (e.g., stonewool cubes or bagged substrates such as coco coir). In some embodiments, the substrate-retention mechanism is adjustable to accept substrates with different dimensions, such as, for example, four- or six-inch cubes or other geometric shapes. The platform can receive and retain the substrate material in a fashion that ensures consistent sensor placement with the substrate. The substrate can remain on the platform throughout the life cycle of the plant to provide continuous in-situ monitoring of the substrate environment.

Embodiments of the station can contain electronics which power and communicate with the sensors, store data, and wirelessly transmit or receive data within a local, wireless network that is cloud-connected for remote data access. The sensors, embedded data recording and storage, and wireless communication can be powered by an embedded, rechargeable battery that is connected to a photovoltaic panel on the exterior of the platform. The photovoltaic panel can provide battery charging as well as measurement of light intensity. The battery and wireless communication make the sensor self-contained and therefore more portable and adaptable than existing devices. Several individual sensor platforms can be installed in a horticulture facility to provide as many sample points as necessary, and all platforms can communicate through a single wireless network and connected to a web-deployed front-end interface.

One aspect of the disclosure relates to a plant substrate sensor station having a housing with a platform to contact a vertically-facing surface of a plant substrate material and with a sensor retainer to retain a sensor probe in a vertical orientation through the vertically-facing surface of the plant substrate material mounted to the housing. The station can also include an electronic station mounted to the housing and configured to receive a signal from the sensor probe. The vertical orientation of the sensor probe can ensure penetration of the probe perpendicular to vertical gradients in the substrate material and can therefore ensure a consistent and easily repeatable vertical depth of insertion in similar substrates for other stations. Accordingly, the measurements of the stations are more readily compared to each other and to historical data for the improvement of irrigation schedules, fertilization schedules, and adjustments to pH, lighting conditions, and other factors.

Another aspect of the disclosure relates to a plant substrate sensor station comprising a base platform, wherein a plant substrate material is contactable by the base platform while the plant substrate material is in a substrate support zone adjoining the base platform. A sensor probe can be mounted to the base platform with the sensor probe having an elongated transducer extending perpendicular to the base platform and vertically into the substrate support zone. Horticultural substrates can have various sizes and shapes yet can have sensor equipment consistently installed within the zones in which the substrates reside. In some embodiments, a sensor station can include a support stand that contacts side surfaces of the plant substrate material to secure the material in position near the sensor station (e.g., on top of the sensor station). This can help improve the portability of the station while also helping to align the substrate material relative to the sensor probe in a manner that improves sensor insertion consistency.

The present description provides examples, and is not limiting of the scope, applicability, or configuration set forth in the claims. Thus, it will be understood that changes may be made in the function and arrangement of elements discussed without departing from the spirit and scope of the disclosure, and various embodiments may omit, substitute, or add other procedures or components as appropriate. For instance, the methods described may be performed in an order different from that described, and various steps may be added, omitted, or combined. Also, features described with respect to certain embodiments may be combined in other embodiments.

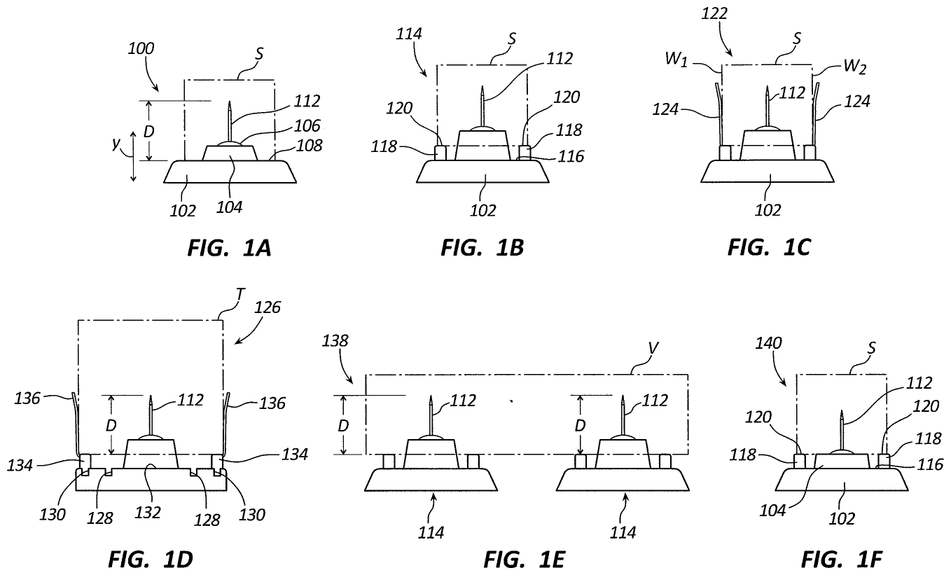

FIGS. 1A-1F schematically illustrate features of sensor stations of the present disclosure. The sensor stations shown in these figures are shown in side view. Features and elements of the individual sensor stations shown in these figures can be implemented in other embodiments of the sensor stations.

FIG. 1A illustrates a schematic diagram of a sensor station 100 according to an embodiment of the present disclosure. The sensor station 100 can comprise a base housing 102 having a sensor retainer portion 104 in which a sensor 106 is positioned. The base housing 102 can also comprise a support surface 108 configured to support a vertically-facing surface of a plant substrate material S mounted to the base housing 102 (e.g., the downward-facing surface of substrate material S). The sensor 106 can have a probe 112 configured to extend vertically upward into the plant substrate material S to a predefined distance D measured relative to the bottom surface of the plant substrate material S. The plant substrate material S is shown in broken lines in FIG. 1A to indicate that its size and shape can vary relative to the sensor station 100.

The base housing 102 can comprise an electronics station (not shown) in electronic communication with the sensor 106. The electronics station can provide power to the sensor station 100 and can control the operation of the sensor 106. In some embodiments, the electronics station can provide electronic communication to a separate sensor station or to an external network location. The base housing 102 can be a molded sensor base that houses or supports electronics, a battery, an antenna, a renewable power source (e.g., a photovoltaic panel or other solar panel), and one or more sensors.

The sensor retainer portion 104 can comprise an opening or void in which one or more sensors (e.g., sensor 106) can be retained or on which they can reside on the base housing 102. The sensor retainer portion can therefore comprise various grips, clamps, openings, apertures, recesses, or similar mechanisms or features configured to hold a sensor 106 in place relative to the base housing 102. The sensor retainer portion 104 can beneficially be designed to hold onto the sensor 106 with sufficient force to prevent the sensor 106 from being dislodged from the sensor retainer portion 104 when force is applied to the plant substrate material S to remove it from the sensor station 100. In some embodiments, as shown in FIG. 1A, the sensor retainer portion 104 can be at least partially inserted into the plant substrate material S and can therefore at least partially penetrate through a surface (e.g., the bottom surface) of the plant substrate material S. In some cases, the plant substrate material S comprises a groove or recess in its station-facing surface and into which the sensor retainer portion 104 can extend. In some cases, the plant substrate material S is supported by a top surface of the sensor retainer portion 104 or by a top surface of a portion of the sensor 106 instead of, or in addition to, being supported by the support surface 108 of the base housing 102.

The sensor 106 can comprise a device used to measure a physical characteristic of the plant substrate material S. The sensor 106 can therefore include one or more transducers such as thermometers, water content sensors, electrical conductivity sensors, water activity sensors, pH sensors, or other related devices used to measure properties of the plant substrate material S. As used herein, measurement or sensing of a property of the “plant substrate material” or “substrate material” includes measurement or sensing of a property of a plant or fluid located in the substrate material, such as a plant or fluid held by a stonewool cube or aggregate of peat or coco coir. The sensor 106 can beneficially comprise a water content/electrical conductivity/temperature sensor probe configured to measure water content, electrical conductivity, and temperature using a single device.

The sensor probe 112 can comprise at least one spike, stick, blade, ridge, tube, or other elongated transducer component configured to penetrate the plant substrate material S and to be positioned therein in a substantially vertical orientation (e.g., parallel to the Y-axis in FIG. 1A). Thus, the plant substrate material S can surround the sensor probe 112 without an air gap or other open space between the probe 112 and the substrate material. The lack of an air gap can improve the reliability and consistency of the measurement made by the probe 112 in the substrate material.

In some embodiments, the sensor probe 112 can include multiple elongated devices extending into the plant substrate material S. In some embodiments, the elongated transducer components can extend into the plant substrate material S along a partially vertical, partially horizontal direction. In this case, the sensor probe 112 can still be configured to extend to a consistent point within a plant substrate material S, such as to a predetermined distance from a point of insertion at the bottom surface of the plant substrate material S.

The support surface 108 can comprise a generally flat, horizontal surface configured to support a bottom surface of the plant substrate material S. The support surface 108 can be water-tight in a manner that prevents water or other fluids passing through the substrate material from penetrating through the support surface 108. In some embodiments, the support surface 108 can comprise a channel or groove for channeling water or other fluids away from portions of the base housing 102 or away from the plant substrate material S. The support surface 108 can contact the bottom surfaces of the plant substrate material S without a gap or space between the surfaces in a manner providing support across the entire underside of the substrate material. In some embodiments, the sensor station 100 can be placed on a top surface of the plant substrate material S, and the sensor probe 112 can extend into the plant substrate material S from the top surface.

FIG. 1B shows another embodiment of a sensor station 114 wherein the plant substrate material S is spaced away from a top surface 116 of the base housing 102. Accordingly, a set of spacers 118 can support the bottom of the plant substrate material S. The set of spacers 118 can be referred to as a support platform or support stand for the plant substrate material S. The set of spacers 118 can support outer portions of the plant substrate material S such as the corners or perimeter thereof. The set of spacers 118 can therefore permit water and air flow beneath the plant substrate material S for aeration and drainage purposes. Accordingly, the set of spacers 118 can comprise a set of top surfaces 120 vertically spaced above the top surface 116 of the base housing 102 and the bottom surface of the plant substrate material S can be spaced away from the top surface 116 of the base housing. In an example embodiment, the set of spacers 118 comprises four spacers, wherein each of the spacers is configured to be positioned under a corner portion of the plant substrate material S. In another embodiment, a spacer is implemented wherein the spacer comprises one or more internal apertures or openings configured to drain fluids through a center area within the spacer. The spacer can be a single piece extending around multiple sections of a bottom perimeter of the plant substrate material S.

FIG. 1C shows yet another embodiment of a sensor station 122 that further comprises a set of support arms 124. The set of support arms 124 can contact opposite-facing, laterally-facing side surfaces of the plant substrate material S (e.g., side surfaces W₁ and W₂). The set of support arms 124 can help orient the assembly of the plant substrate material S relative to the sensor station 122. The set of support arms 124 can also provide a laterally-inward-directed force to the side surfaces (e.g., W₁ and W₂) that helps to keep the plant substrate material S in position after installation. In some embodiments, the sensor station 122 comprises support aims 124 configured to contact each corner section of the plant substrate material S. The set of support arms 124 can extend from a set of spacers 118 and/or from the base housing 102. The set of support arms can be referred to as elongated fingers that are elongated in a vertical direction or parallel to the sensor probe 112.

FIG. 1D illustrates another embodiment of a sensor station 126 that is configured to support multiple sizes of plant substrate materials. The base housing 102 can comprise a set of mounting points 128, 130 in a top surface 132 thereof configured to releasably receive a set of movable spacers 134. The mounting points 128, 130 are represented as grooves or apertures, but they can take on other shapes such as interlocking parts, magnetic bindings, and similar elements. The movable spacers 134 can therefore be positioned at mounting points 128, mounting points 130, or combinations thereof depending on the size and positioning of the plant substrate material being used. While the spacers 134 are located at the inner mounting points 128, a smaller plant substrate material (e.g., S) can be supported with support arms 136 contacting its outer side surfaces, and while the spacers 134 are located at the outer mounting points 130, a larger plant substrate material T can be supported with the support arms 136 on its outer side surfaces (as shown in FIG. 1D). The depth of insertion D of the sensor probe 112 can be consistent whether the smaller or larger plant substrate material is being used and whether the spacers 134 are at the inner or outer mounting points 128, 130. The mounting points 128, 130 can comprise a number of predetermined positions (e.g., the inner position represented by points 128 and the outer position represented by points 130) or can comprise a plurality of infinitely variable or infinitely adjustable positions, wherein any size of plant substrate material within the range of infinitely adjustable positions can be supported. For example, a mounting point can be positioned on a movable platform or other support member that can be repositioned on the base housing 102 to accommodate different sizes of plant substrate materials.

FIG. 1E shows a system 138 wherein multiple sensor stations 114 are used to support an elongated plant substrate material V. Accordingly, multiple sensor stations can be used to measure one or more properties of a single plant substrate material. Additionally, the shape of the plant substrate material can be larger than the sensor stations, and the sensor probes 112 can be inserted at positions that are not horizontally centered in the plant substrate material V. Multiple sensor probes 112 can be configured to penetrate the plant substrate material V up to a single, consistent depth of insertion D that is common to all of the sensor stations 114.

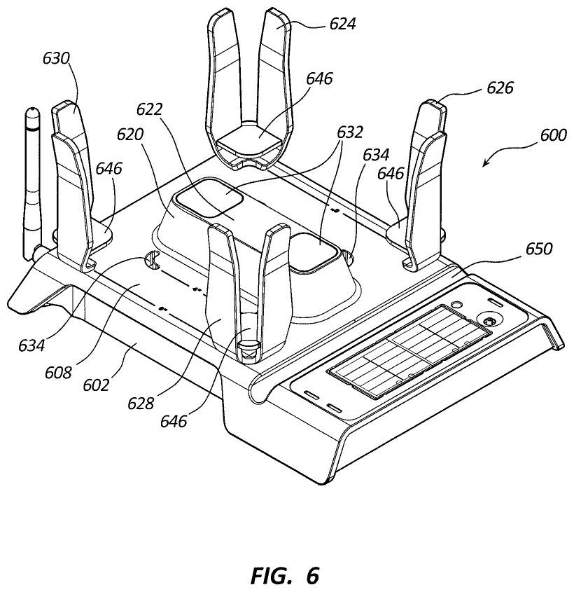

As shown in FIG. 1F, a sensor station 140 can comprise a sensor retainer portion 104 that has a height relative to the top surface 116 of the base housing 102 that is substantially equal to top surfaces 120 of the set of spacers 118. Accordingly, the sensor retainer portion 104 can contact the bottom surface of the plant substrate material S without penetrating into the material. The sensor probe 112 can still penetrate into the material above the sensor retainer portion 104. In some embodiments, the sensor station 140 of FIG. 1F lacks the set of spacers 118, and the plant substrate material S can rest on top of the sensor retainer portion 104 alone. In some embodiments, a set of sensor pads are positioned at the top of the sensor retainer portion instead of, or in addition to, the elongated sensor probe 112. See FIG. 6. The sensor pads can each have a substantially flat top surface that faces upward to contact a bottom surface of the plant substrate material S. The sensor pads can therefore have top surfaces in contact with, and coplanar with, the bottom surface of the plant substrate material S. The top surfaces of the sensor pads can be parallel to support surface 108.

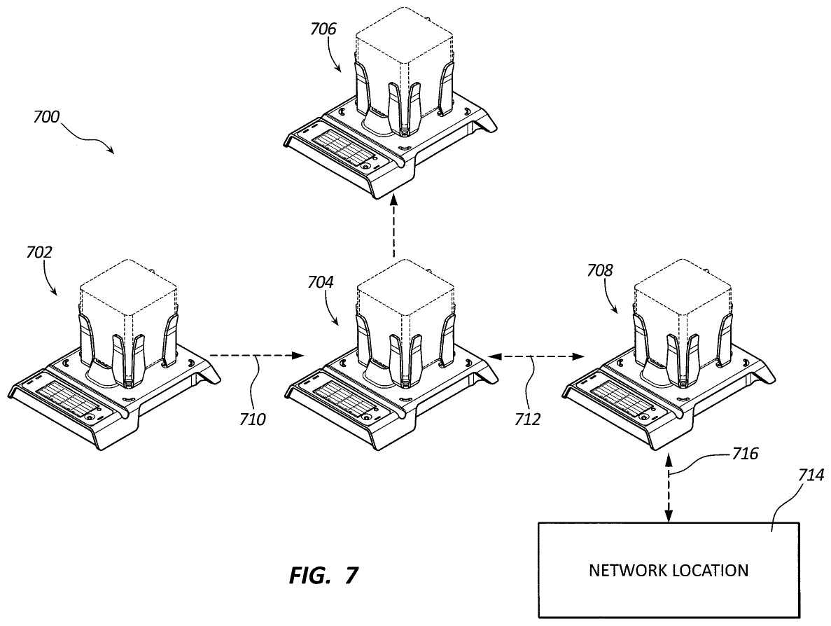

The sensor stations 114 can be in electrical communication with each other. A wired or wireless connection interface can allow the sensor stations 114 to relay sensor signal information, status information, and other information to each other or to a third device. For example, the sensor stations can comprise antennae (not shown) for wireless communication with each other using a wireless network protocol such as WI-FI®, BLUETOOTH®, ZIGBEE®, and related connection types, as explained in further detail in connection with FIG. 7 below.

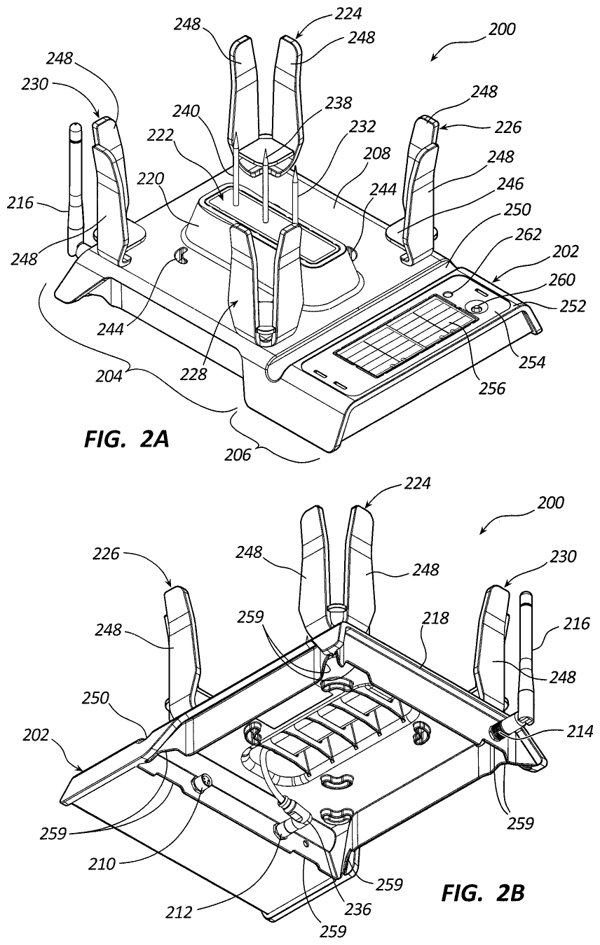

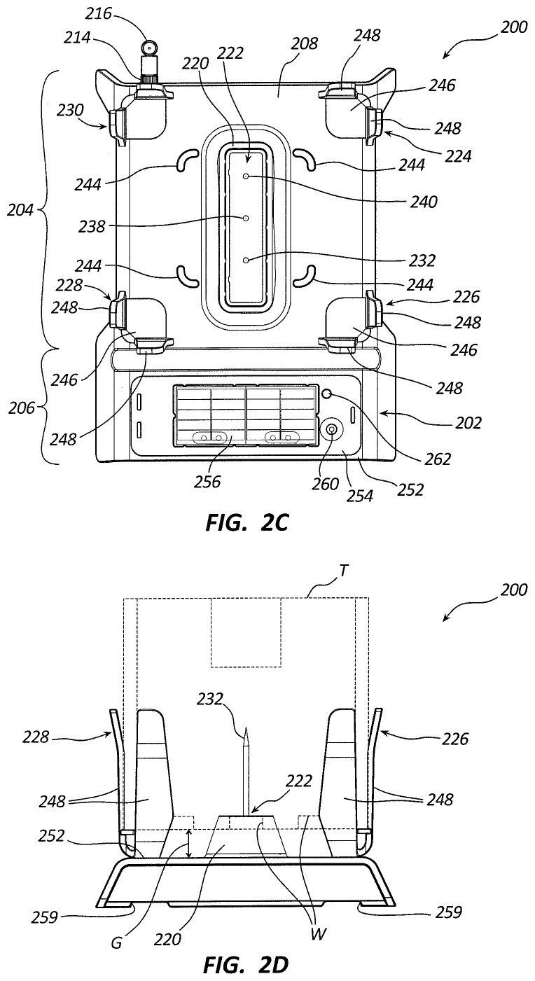

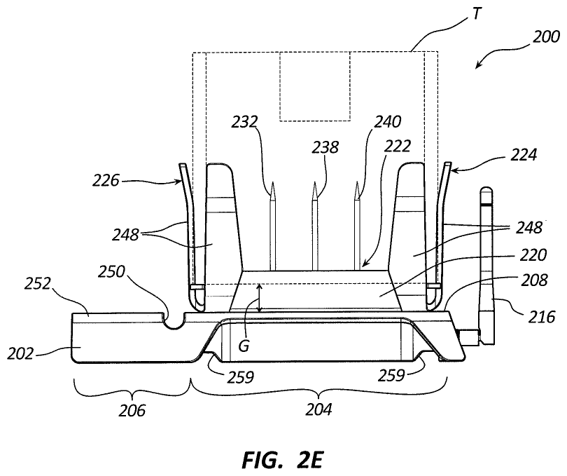

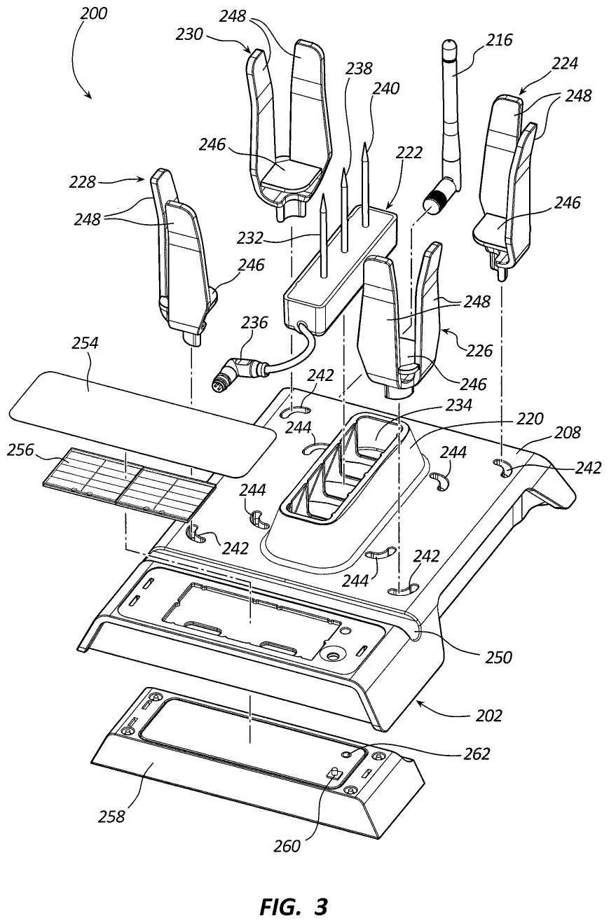

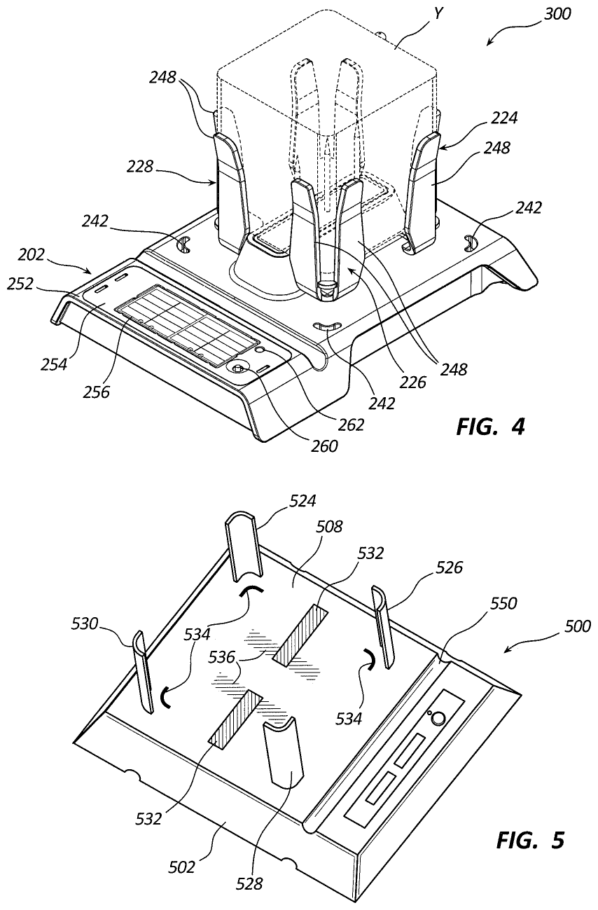

A particular embodiment of a sensor station 200 incorporating various aspects of the sensor stations 100, 114, 122, 126, 140 of FIGS. 1A-1F is shown in FIGS. 2A-2E. FIG. 2A shows an isometric view generally showing the top and sides of the sensor station 200, FIG. 2B shows an isometric view generally showing the bottom and sides of the sensor station 200, FIG. 2C is a top view, FIG. 2D is a front view, and FIG. 2E is a right side view. FIG. 3 is an exploded view, and FIG. 4 is an alternate configuration of the sensor station 200.

The sensor station 200 can comprise a base housing 202 having a substrate platform portion 204 and an interface portion 206. See FIGS. 2A, 2C, and 2E. The substrate platform portion 204 can be configured to be positioned under a plant substrate material (e.g., plant substrate material T in FIGS. 2D and 2E). The substrate platform portion 204 can have a generally square and planar top surface 208. In some embodiments, the top surface 208 can have a different shape (e.g., rectangular or circular), such as a shape corresponding to a general shape of the plant substrate material under which it is configured to be positioned. The top surface 208 can be water-tight and can prevent fluids from passing into contact with electrical connectors 210, 212, 214 of the electronics in the interface portion 206 and the antenna 216. See FIG. 2B. In some embodiments, the top surface 208 comprises a ledge portion 218 overhanging one or more electrical connectors (e.g., connector 214 in FIG. 2B).

The substrate platform portion 204 can also comprise a sensor retainer 220 configured to support and retain at least one sensor 222. The substrate platform portion 204 can also include a set of substrate support stands 224, 226, 228, 230 configured to engage the plant substrate material T (see FIGS. 2D-2E). The sensor retainer 220 can have outer walls that extend vertically upward from the top surface 208 of the substrate platform portion 204. The outer sidewalls of the sensor retainer 220 can also have a sloped grade, as shown in FIG. 2D, wherein they help flush fluids away from the sensor 222 and across the top surface 208:

The raised nature of the sensor retainer 220 can help retain the sensor 222 at a raised position relative to the top surface 208 of the substrate platform portion 204. This can allow the sensor station 200 to hold a plant substrate material T raised above or spaced vertically away from the top surface 208, as indicated by gap G in FIGS. 2D-2E, without the sensor 222 being spaced away from the plant substrate material T. The gap G can allow fluids and debris to pass below the plant substrate material T, thereby improving airflow and reducing stagnant water at the underside of the plant substrate material T. Complete coverage of the sensor 222, meaning there are no gaps or spaces between the sensor 222 and the substrate material, can improve the consistency and accuracy of the sensor's output. In some embodiments (not shown), a sensor retainer can be provided that supports a sensor at a vertical level even with, or below, the top surface 208, thereby eliminating the gap G.

The top of the sensor retainer 220 shown in FIGS. 2D and 2E can penetrate partially into the bottom half of the plant substrate material T. This can help ensure that any gaps between the bottom of the plant substrate material T are eliminated upon installation of the plant substrate material T to the sensor station 200, especially if there are grooves or recesses (e.g., grooves W in FIG. 2D) in the bottom of the plant substrate material T. In some embodiments, the bottom surface of the plant substrate material T is configured to rest on a surface of the sensor 222 and the sensor retainer 220 with only a probe (e.g., probe 232) or other relatively elongated and narrower portion of the sensor 222 penetrating the plant substrate material T, as shown in the sensor station 140 of FIG. 1F. In some embodiments, a flat sensor pad can be used in place of one or more probe 232, such as sensor pads 532/536 in FIG. 5 or sensor pads 632 in FIG. 6. Accordingly, a sensor pad can be configured to rest against a side surface of the plant substrate material T to transduce properties of the substrate material.

The sensor retainer 220 can comprise a generally rectangular aperture or inner recess 234 in which the sensor 222 is positioned. The aperture or inner recess can be referred to as a sensor-shaped opening or aperture in the sensor station 200 since it can surround and support the sensor 222 on all of its lateral sides. The inner recess 234 can support the sides and/or bottom of the sensor 222 while allowing an electrical connector 236 of the sensor 222 to connect to an electrical connector 212 of the base housing 202. The inner recess 234 can have a top opening through which one or more probes (e.g., probes 232, 238, 240) extend vertically into a space above the sensor retainer 220 and above the top surface 208. In some embodiments, the sensor retainer 220 lacks sidewalls and only provides an inner recess 234 in the top surface 208 in which the sensor 222 is positioned.

The sensor retainer 220 is centered on the top surface 208 and centrally positioned relative to the substrate support stands 224, 226, 228, 230. Accordingly, the substrate support stands 224, 226, 228, 230 surround and are positioned equidistant from the sensor retainer 220. The substrate support stands 224, 226, 228, 230 therefore guide and hold the center of a plant substrate material T above or on top of the sensor retainer 220. The sensor retainer 220 (or sensor 222) and the plant substrate material T can therefore have aligned central vertical axes. In this manner, multiple plant substrate material blocks attached to multiple sensor stations 200 will have a probe positioning and depth that is consistent and equal. Extending into the plant substrate material at the same depth and position from case to case can reduce measurement variation that can result from sensors being positioned in different parts of different substrates in a horticulture facility. Thus, more consistent and reliable readouts can be obtained by guiding the substrate material into the same position and orientation relative to the sensor 222 using the substrate support stands 224, 226, 228, 230 and sensor retainer 220.

The vertical orientation of the probes (e.g., 232, 238, 240) even further reduces variation by penetrating through vertical material property gradients in a substrate. In the substrate material, properties such as water content can strongly vary based on the vertical depth in which the water content is measured, so consistent vertical penetration depth across multiple stations strongly reduces measurement error and improves consistency. The relative positioning of the substrate support stands 224, 226, 228, 230 and sensor retainer 220 also reduces horizontal variation in probe positioning, thereby reducing error and inconsistency caused by horizontal gradients.

The sensor 222 can comprise one or more sensor or transducer devices configured to measure or sense characteristics and properties of the plant substrate material T. In some embodiments, the sensor 222 comprises one or more sensors to measure water activity, electrical conductivity, temperature, pH, water drainage volume, other similar properties or characteristics, or combinations thereof. The sensor 222 can comprise one or more probes 232, 238, 240 that are configured to vertically extend into the plant substrate material T. The probes 232, 238, 240 can provide sensing for different properties or characteristics, such as a sensor 222 with probes 232, 238, 240 for measuring water content, electrical conductivity, and temperature. In some embodiments, the sensor 222 is removable from the inner recess 234 and can be exchanged for another sensor or another type of sensor. This can beneficially allow the sensor station 200 to be repaired, modified, and upgraded.

The substrate support stands 224, 226, 228, 230 can be retained in apertures or recesses 242, 244 in the top surface 208. See FIG. 3. The substrate support stands 224, 226, 228, 230 can therefore be movable and repositionable relative to the base housing 202 and the plant substrate material T. In some embodiments, the substrate support stands 224, 226, 228, 230 are movable between two distinct configurations, such as a first configuration with the substrate support stands 224, 226, 228, 230 in the positions of recesses 242 and a second configuration in the positions of recesses 242. See FIGS. 2A and 4. In some embodiments, the substrate support stands 224, 226, 228, 230 can be positioned in a plurality of different configurations, including, for example, a mixture of usage of the different recesses 242, 244 or in an infinitely adjustable retainer on the top surface 208. The substrate support stands 224, 226, 228, 230 can interlock with the recesses 242, 244 or can be otherwise connected to the base housing 202 such as by being co-molded, formed with, attached to, or otherwise mounted on the base housing 202.

In various configurations, the substrate support stands 224, 226, 228, 230 can support and retain various sizes and shapes of plant substrate materials. For example, in FIG. 2E, the substrate material T is a cube with about six-inches of length, width, and height, and the substrate support stands 224, 226, 228, 230 are positioned in the outer recesses 242 in a manner supporting the bottom corners and lateral side surfaces near the edges of a cube of that size. In FIG. 4, the substrate support stands 224, 226, 228, 230 are positioned in the inner recesses 244 in a manner supporting the bottom corners and lateral side edges of a plant substrate material Y having a cube shape with about four-inch side dimensions. In other configurations, the recesses and substrate support stands can be configured to support other cube sizes, rectangular-prism-shaped blocks, cylindrical blocks, spherical blocks, or other shapes.

The substrate support stands 224, 226, 228, 230 can each comprise a support surface 246 and one or more finger portions 248. The support surfaces 246 can be configured to contact a bottom surface of the plant substrate material. They can therefore give support to the substrate material and provide a bottom stop for the movement of the substrate material when it is inserted onto the sensor station 200. The support surfaces 246 can be parallel to the top surface 208 of the base housing 202 and can be spaced vertically above the top surface 208, thereby defining the size of the gap G. The support surfaces 246 can be positioned at a lower vertical position than the top end of the sensor retainer 220. See FIGS. 2D and 2E. Accordingly, the plant substrate material can be pressed down over the sensor retainer 220 and sensor 222 until it contacts the support surfaces 246. Contact with the support surfaces 246 can indicate that the substrate material has been completely inserted into the substrate support stands 224, 226, 228, 230.

Four substrate support stands 224, 226, 228, 230 are shown in the embodiment of FIGS. 2A-4. In some embodiments, another number of support stands can be used, such as two support stands extending parallel to each other on each opposite lateral side of the sensor retainer 220. In another embodiment, a single substrate support stand is used that extends around three or four sides of the sensor retainer 220 with a central opening (i.e., in a U- or ring-shaped configuration around the sensor retainer 220).

The substrate support stands 224, 226, 228, 230 can each comprise two finger portions 248. The finger portions 248 can be arranged substantially perpendicularly relative to each other on each substrate support stand 224, 226, 228, 230, thereby allowing the finger portions 248 to simultaneously contact two adjacent side surfaces of the plant substrate material. The two adjacent side surfaces can be flat side surfaces that adjoin an edge positioned between the finger portions 248. The finger portions 248 can be vertically elongated and can be blade- or panel-shaped, wherein they have a greater lateral width than thickness. The increased lateral width can allow the finger portions 248 to support plant substrate materials that are misshapen and can help prevent the finger portions 248 from cutting into or penetrating the substrate material when applying pressure to it. The finger portions 248 can also have top ends that flare laterally outward and away from the substrate material. See FIGS. 2D and 2E. The flared ends can act as a guide or funnel to assist a user in inserting the plant substrate material into the substrate support stands 224, 226, 228, 230. The flared ends can also provide a space between the substrate material and the finger portions 248 so that the top ends of the finger portions 248 can be conveniently pulled away from the substrate material when removing or adjusting the substrate material.

The finger portions 248 can be configured to resiliently flex outward as the plant substrate material T is inserted into its space within the finger portions 248. Accordingly, the finger portions 248 can apply an inwardly-directed pressure to the sides and corner portions of the plant substrate material. This pressure can help keep the substrate material from moving when the sensor station 200 is moved and operated, thereby also keeping the sensor 222 properly positioned in the substrate material.

In some configurations, at least some of the finger portions 248 can be omitted, thereby allowing a plant substrate material to rest on the support surfaces 246 without being contacted on four lateral sides. For example, the finger portions 248 can be configured to only contact one or both of the opposite front and back surfaces of a plant substrate block. The finger portions 248 can also be entirely omitted, thereby allowing the substrate material to be positioned with the sensor 222 at any lateral position in the substrate material. See, e.g., FIGS. 1A, 1B, and 1E.

The platform portion 204 and the interface portion 206 can have a groove, aperture, or channel 250 positioned between their top surfaces. See FIGS. 2A and 2E. When water or other fluids flow from the platform portion 204 toward the interface portion 206, the channel 250 can redirect the fluid so as to limit the amount of flow that reaches the interface portion 206 on the other side of the channel 250. The channel 250 can thereby assist in keeping fluids and debris carried by fluids from collecting on the interface portion 206.

The interface portion 206 can comprise a top surface 252 within which a transparent panel 254 is positioned and below which a generator 256 and an electronics station 258 are positioned. See FIGS. 2C and 3. The transparent panel 254 can be water-tightly sealed to the interface portion 206 to resist penetration of liquid into the interface portion 206 through the top surface 252. The transparent panel 254 can allow light to pass to the generator 256, which can be a solar/photovoltaic generator. The generator 256 can provide power to the sensor station 200 in a manner reducing or eliminating a need for an outside power source (e.g., a connection to an electrical utility distribution grid). The generator 256 can also be used for energy harvesting, wherein the generator 256, in conjunction with an energy storage device (e.g., a battery) in the electronics station 258 can store energy produced by the generator 256. The generator 256 can allow the sensor station 200 to operate continuously and indefinitely as long as there is a baseline amount of light intensity (e.g., regular sunlight or indoor light exposure normally used to grow plants in a horticulture environment). In some embodiments, the generator 256 can comprise an external connection to a wind generator or another type of power generator. In some embodiments, the generator 256 can be omitted, and the sensor station 200 can be connected to an external power source.

A solar or photovoltaic (PV) generator 256 and electronics station 258 can be used as a light sensor for the sensor station 200. In some embodiments, a battery (e.g., Li-ion cell) charge controller can be used to determine light intensity based on output of a monocrystalline photovoltaic cell. The PV generator can generate current linearly proportional to power density. The electronics can therefore include a current-sensing resistor that can be amplified to provide a voltage output corresponding to light intensity incident on the PV panel. The electronics can also include features to limit over- or under-charge of a battery or other energy storage device powered by the PV panel.

The electronics station 258 can comprise a user interface. In the embodiment of FIGS. 2A-4, the user interface comprises a button 260 and an indicator light 262. The button 260 and indicator light 262 can be used to provide or receive information for a user, such by providing a status of the sensor station 200, a warning, a sensor measurement, or receiving instructions regarding which sensors to operate, network connectivity settings, power on/off, etc. The button 260 and indicator light 262 are respectively input and output devices. Other input and output devices can be used in place of, or in addition to, the button 260 and indicator light 262.

The electronics station 258 can comprise electrical connectors 210, 212. See FIG. 2B. One electrical connector 210 can be used to connect the electronics station 258 to an external device and can be, for example, an M8, CAT5, PS/2, USB, or other similar connector. Another electrical connector 212 can connect to the sensor 222 at its electrical connector 236. The electrical connectors 210, 212 can be positioned under the top surfaces 208, 252 of the sensor station 200 to limit their exposure to fluids, dust, and debris. The base housing 202 can comprise a set of recesses or similar passageways 259 to allow fluids and debris to pass underneath and escape the area under the sensor station 200. See FIG. 2B.

The electronics station 258 can also be in electrical communication with the electrical connector 214 for the antenna 216 in order to wirelessly connect to other external devices. For example, a wire (not shown) can link the electronics station 258 to the electrical connector 214. The electronics station 258 can also comprise a modem or other network connectivity device (not shown) that, in combination with the antenna 216 or another network adapter (e.g., a 2.4 GHz wireless radio adapter), can allow the sensor station 200 to electronically communicate with an external device. In some embodiments, the network connectivity device can comprise a transceiver, wherein the sensor station 200 can receive and send information via the network connectivity device. In this manner, the sensor station 200 can receive operating instructions via the network connectivity device and can send data (e.g., sensor measurement data or station operating status data) via the network connectivity device.

FIG. 5 shows an isometric view of another embodiment of a sensor station 500 according to the present disclosure. The sensor station 500 can have a base housing 502 with a planar top surface 508 lacking a protruding sensor retainer 220. Accordingly, the top surface 508 of the base housing 502 can be substantially flat. The base housing 502 can have the features and inner components of base housing 202.

The top surface 508 can comprise flat sensor pads 532 that are substantially coplanar with the top surface 508. The set of substrate support stands 524, 526, 528, 530 can extend directly from the top surface 508 without an intervening support surface 246 (or with a support surface that is substantially flush with the top surface 508).

In this manner, the sensor station 500 can operate with a plant substrate material T that rests on the top surface 508 and that rests on top of flat sensor pads 532. This can be beneficial when a gap (e.g., G) is not desired between the substrate material and the top surface 508. The substrate support stands 524, 526, 528, 530 can still retain the plant substrate material T in place on the top surface 508 similar to the substrate support stands 224, 226, 228, 230 of station 200. The substrate support stands 524, 526, 528, 530 can be removed and repositioned as well. For example, the substrate support stands 524, 526, 528, 530 can be positioned in inner recesses 534 to support and center a smaller substrate material (e.g., substrate material S). With the substrate support stands 524, 526, 528, 530 removed, the sensor station 500 can have a very low profile and can therefore be packaged, stored, or transported more easily.

The sensor pads 532 can comprise a flat stainless steel pad or similar conductor or radiator for sensing electrical conductivity, water content, temperature, or other properties of a plant substrate material T contacting the pads 532. One or more sensor pads 532 can be used. In sensor station 500, two sensor pads 532 are implemented in a manner parallel to a channel (e.g., 550) in the base housing 502. The sensor pads 532 can alternatively be oriented perpendicular to the channel in the base housing 502, as shown by pads 536. The orientation of the pads 532/536 can affect the amount of surface area of the pads that contacts the plant substrate material (which can be associated with the strength of the signal detected by the sensors), the size and shape of the sensor components within the base housing 502, and the types and shapes of plant substrate materials that can be monitored by the station 500. For example, a plant substrate material having bottom grooves W can be oriented relative to the station 500 so that pads 532 are aligned with and in contact with portions of the plant substrate material that reach and contact the top surface 508. In some embodiments, sensor pads 532 can be implemented, in some embodiments, pads 536 can be implemented, and in some embodiments, both pads 532, 536 are implemented.

In some cases, elongated probes (e.g., one or more probes 232, 238, 240) can be added to the station 500 and can extend vertically from the top surface 508 and into a space above the top surface 508 where the plant substrate material T is configured to be positioned (i.e., between the substrate support stands 524, 526, 528, 530). Thus, the probes do not need to be spaced from the top surface 508 by a sensor retainer 220 or similar structure. Sensor pads 532/536 and probes 232, 238, 240 based on and extending from the top surface 508 can be implemented in the sensor station 500 separately or together.

FIG. 6 shows an isometric view of another embodiment of another sensor station 600 according to the present disclosure. The sensor station 600 can have a base housing 602 with a planar top surface 608 and a protruding sensor retainer 220. The top surface 608 of the base housing 602 can be substantially flat, and a top surface 622 of the sensor retainer 220 can be substantially flat and parallel to the top surface 608 of the base housing 602. The base housing 602 can have the features and inner components of base housings 202 or 502.

The sensor retainer 620, or a sensor positioned within it, can comprise flat sensor pads 632 that are substantially parallel to the top surface 608 and are configured to be parallel to, and in contact with, a plant substrate material positioned on the sensor retainer 620. The set of substrate support stands 624, 626, 628, 630 can extend from the top surface 608, and each can have a support surface 646 that is substantially parallel to the top surface 608 and the sensor pads 632. In some embodiments, the support surface 646 is positioned at the same vertical distance from the top surface 608 as the sensor pads 632.

In this manner, the sensor station 600 can operate with a plant substrate material T that rests on the top surface 622 and that rests on top of support surfaces 646. This can be beneficial when a gap (e.g., G) is desired between the substrate material and the top surface 608. The substrate support stands 624, 626, 628, 630 can also retain the plant substrate material T centered in place on the top surface 608 similar to the substrate support stands 224, 226, 228, 230 of station 200. The substrate support stands 624, 626, 628, 630 can be removed and repositioned as well. For example, the substrate support stands 624, 626, 628, 630 can be positioned in inner recesses (e.g., 634) to support and center a smaller substrate material (e.g., substrate material S). With the substrate support stands 624, 626, 628, 630 removed, the sensor station 600 can have a low profile and can therefore be packaged, stored, or transported more easily. The lack of an elongated spike or probe extending from the sensor retainer 620 can greatly reduce the overall height of the sensor station 600.

The sensor pads 632 can comprise a flat stainless steel pad or similar conductor or radiator for sensing electrical conductivity, water content, temperature, or other properties of a plant substrate material T contacting the pads 632. One or more sensor pads 632 can be used. In sensor station 600, two sensor pads 632 are implemented on the top surface 622 along a line perpendicular to the channel 650 in the housing 602. The sensor pads 632 can alternatively be oriented along a line parallel to the channel in the base housing 602. The orientation of the pads 632 can affect the amount of surface area of the pads that contacts the plant substrate material (which can be associated with the strength of the signal detected by the sensors), the size and shape of the sensor components within the base housing 602, and the types and shapes of plant substrate materials that can be monitored by the station 600. For example, a plant substrate material having bottom grooves W can be oriented relative to the station 600 so that pads 632 are aligned with and in contact with portions of the plant substrate material that reach and contact the top surface 608.

In some cases, elongated probes (e.g., one or more probes 232, 238, 240) can be added to the station 600 and can extend vertically from top surfaces 608 or 622 and into a space above the top surface 608 where the plant substrate material T is configured to be positioned (i.e., between the substrate support stands 624, 626, 628, 630). Thus, the probes can be, but do not need to be, spaced from the top surface 608 by a sensor retainer 220/620 or similar structure. Sensor pads 632 and probes 232, 238, 240 based on and extending from top surfaces 608 or 622 can be implemented in the sensor station 600 separately or together.

In some embodiments, the sensor station 200 can send data to, and receive data from, another external sensor station. FIG. 7 is a diagram of a network 700 including a set of sensor stations 702, 704, 706, 708 in communication with each other. At least one of the sensor stations can be configured to store or transmit data received from other sensor stations. For example, station 704 can receive data from station 702, as indicated by arrow 710, and can store that data or transmit it, as indicated by arrow 712, to another station 708. At least one station 708 can be connected to an external network location 714. The external network location 714 can comprise a computer or other electronic device configured to store data from the sensor stations 702, 704, 706, 708. The network location 714 can also comprise a device to generate instructions to send to sensor stations configured to receive them via a connection to the network location 714 (e.g., station 708, as indicated by arrow 716) or via a connection to another sensor station, such as station 704 which is connected to and can receive data from station 708. Station 706 can receive data from station 704 as well. In some embodiments, all stations 702, 704, 706, 708 are capable of two-way communication with other stations. Thus, the network 700 can comprise a multi-hop wired or wireless mesh network. The communications can be private and encrypted at each station 702, 704, 706, 708. This network configuration can be beneficial where typical wireless network connectivity (e.g., WI-FI®, cellular, or other similar wireless network connectivity) is not effective since the stations 702, 704, 706, 708 only need to connect to each other and at least one network location 714 rather than to an external wireless network. The at least one network location 714 may or may not be connected to an external network (e.g., the Internet, a wide area network (WAN), local area network (LAN), or an intranet), and if it is, the entire network 700 only needs one point of connection to that external network rather than a connection at each station 702, 704, 706, 708.

Various inventions have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including:” and “having” come as used in the specification and claims shall have the same meaning as the term “comprising.”

What is claimed is:

1. A plant substrate sensor station, comprising:
a housing including:
a platform to contact a vertically-facing surface of a plant substrate material mounted to the housing; and
a sensor retainer to retain a sensor probe in a vertical orientation through the vertically-facing surface of the plant substrate material mounted to the housing; and
an electronics station mounted to the housing and configured to receive a signal from the sensor probe.

2. The plant substrate sensor station of claim 1, further comprising:
the sensor probe retained by the sensor retainer, the sensor probe electrically connected to the electronics station, the sensor probe extending vertically into a space adjacent to the platform, the space being configured to be occupied by the plant substrate material;
a wireless transceiver connected to the electronics station to transmit the signal from the sensor probe; and
a renewable power generator to provide power to the sensor probe, the electronics station, and the wireless transceiver, the renewable power generator being mounted to the housing, laterally spaced from the platform, and configured to be laterally spaced away from the vertically-facing surface of the plant substrate material when the plant substrate material is mounted to the housing.

3. The plant substrate sensor station of claim 1, further comprising the sensor probe, the sensor probe being retained by the sensor retainer, the sensor probe having an elongated sensor prong extending vertically from the sensor retainer to penetrate the plant substrate material.

4. The plant substrate sensor station of claim 1, wherein the housing comprises a base portion having a second vertically-facing surface, the platform being vertically spaced away from the second vertically-facing surface.

5. The plant substrate sensor station of claim 1, wherein the platform comprises a set of spaced apart posts to contact the plant substrate material.

6. The plant substrate sensor station of claim 5, wherein the sensor retainer is configured to retain the sensor probe centered within the set of spaced apart posts.

7. The plant substrate sensor station of claim 1, wherein the housing includes a finger portion to engage a lateral side surface of the plant substrate material.

8. The plant substrate sensor station of claim 7, wherein the housing includes a second finger portion to engage a second lateral side surface of the plant substrate material.

9. The plant substrate sensor station of claim 1, wherein the housing includes a top surface having a channel positioned between the platform and the electronics station.

10. The plant substrate sensor station of claim 1, wherein the platform is attachable to the housing in at least two discrete positions relative to the sensor retainer.

11. A plant substrate sensor station, comprising:
a base platform, wherein a plant substrate material is contactable by the base platform while the plant substrate material is in a substrate support zone adjoining the base platform; and
a sensor probe mounted to the base platform, the sensor probe having an elongated transducer extending perpendicular to the base platform and vertically into the substrate support zone.

12. The plant substrate sensor station of claim 11, further comprising an electronic receiver in electrical communication with the sensor probe.

13. The plant substrate sensor station of claim 11, further comprising a set of finger portions configured to extend alongside the plant substrate material in a direction substantially perpendicular to the base platform.

14. The plant substrate sensor station of claim 11, wherein the base platform is configured to be centered under the plant substrate material and the elongated transducer is positioned within the base platform.

15. The plant substrate sensor station of claim 14, wherein the sensor probe is centered within the base platform.

16. A plant substrate sensor station, comprising:
a base housing having a top surface configured to be positioned beneath a plant substrate material, the plant substrate material having an outer perimeter and a bottom surface;
a substrate support stand having a first support surface and a second support surface, the first support surface being configured to contact a first side surface of the plant substrate material, the second support surface being configured to contact a second side surface of the plant substrate material, the first side surface being opposite the second side surface; and
a sensor system configured to measure properties of the plant substrate material while the plant substrate material is contacted by the first support surface and the second support surface.

17. The plant substrate sensor station of claim 16, wherein the substrate support stand comprises a set of posts, the set of posts comprising a first post having the first support surface and a second post having the second support surface.

18. The plant substrate sensor station of claim 16, wherein a block of the plant substrate material is vertically insertable into the substrate support stand.

19. The plant substrate sensor station of claim 16, wherein the substrate support stand comprises a set of platform portions configured to support corners of the plant substrate material.

20. The plant substrate sensor station of claim 19, wherein the set of platform portions is vertically spaced away from the top surface of the base housing.
